(19) United States
(12) Patent Application Publication
(10) Pub. No.: US 2011/0300079 A1
(43) Pub. Date: Dec. 8, 2011

(54) COMPOSITIONS FOR USE WITH A SYSTEM FOR IMPROVED COOLING OF SUBCUTANEOUS LIPID-RICH TISSUE

(75) Inventors: Paul W. Martens, Pleasanton, CA (US); John Potosky, San Jose, CA (US)

(73) Assignee: Zeltiq Aesthetics, Inc., Pleasanton, CA (US)

(21) Appl. No.: 13/011,640

(22) Filed: Jan. 21, 2011

Related U.S. Application Data
(60) Provisional application No. 61/297,238, filed on Jan. 21, 2010.

(51) Int. Cl. A61K 31/08; A61P 39/00; A61K 31/79; A61K 31/19; A61K 31/047; A61K 31/10

(52) U.S. Cl. 424/10.1

(57) ABSTRACT

A composition or cryoprotectant for use with a system for improved cooling of subcutaneous lipid-rich tissue of a subject having skin is provided. The cryoprotectant is a non-freezing liquid, gel, or paste for allowing pre-cooling of a treatment device below about 0° C. while preventing the formation of ice thereon. The cryoprotectant may also prevent freezing of the treatment device to the skin and protect tissue of a subject from freezing damage due to, e.g., ice formation. The cryoprotectant can also include an authentication or anti-counterfeiting agent to authenticate an origin of the cryoprotectant, a lubricating agent to increase lubriciousness of the cryoprotectant, and a thixotropic or pseudoplastic agent in an amount effective substantially to render the gel dimensionally stable unless agitated.

### CROSS-REFERENCE TO RELATED APPLICATION(S)

The present application claims priority under 35 U.S.C. §119(e) of U.S. Provisional Application Ser. No. 61/297,238, filed Jan. 21, 2010, the disclosure of which is incorporated herein by reference in its entirety.

The following commonly-assigned U.S. patent applications are incorporated herein by reference in their entirety:

U.S. Patent Publication No. US 2008/0287839 entitled “METHOD OF ENHANCED REMOVAL OF HEAT FROM SUBCUTANEOUS LIPID-RICH CELLS AND TREATMENT APPARATUS HAVING AN ACTUATOR”;

U.S. Pat. No. 6,032,675 entitled “FREEZING METHOD FOR CONTROLLED REMOVAL OF FATTY TISSUE BY LIPOSUCTION”;

U.S. Patent Publication No. US 2007/0255362 entitled “CRYOPROTECTANT FOR USE WITH A TREATMENT DEVICE FOR IMPROVED COOLING OF SUBCUTANEOUS LIPID-RICH CELLS”;

U.S. Pat. No. 7,854,754 entitled “COOLING DEVICE FOR REMOVING HEAT FROM SUBCUTANEOUS LIPID-RICH CELLS”;

U.S. Patent Publication No. US 2008/0077201 entitled “COOLING DEVICES WITH FLEXIBLE SENSORS”;

U.S. Patent Publication No. US 2008/0077211 entitled “COOLING DEVICE HAVING A PLURALITY OF CONTROLLABLE COOLING ELEMENTS TO PROVIDE A PREDETERMINED COOLING PROFILE”;

U.S. Patent Publication No. US 2009/0118722 entitled “METHOD AND APPARATUS FOR COOLING SUBCUTANEOUS LIPID-RICH CELLS OR TISSUE”;

U.S. Patent Publication No. US 2009/0018624 entitled “LIMITING USE OF DISPOSABLE SYSTEM PATIENT PROTECTION DEVICES”;

U.S. Patent Publication No. US 2009/0018623 entitled “SYSTEM FOR TREATING LIPID-RICH REGIONS”;

U.S. Patent Publication No. US 2009/0018625 entitled “MANAGING SYSTEM TEMPERATURE TO REMOVE HEAT FROM LIPID-RICH REGIONS”;

U.S. Patent Publication No. US 2009/0018627 entitled “SECURE SYSTEM FOR REMOVING HEAT FROM LIPID-RICH REGIONS”;

U.S. Patent Publication No. US 2009/0018626 entitled “USER INTERFACES FOR A SYSTEM THAT REMOVES HEAT FROM LIPID-RICH REGIONS”;

U.S. Pat. No. 6,041,787 entitled “USE OF CRYOPROTECTIVE AGENT COMPOUNDS DURING CRYOSURGERY”;

U.S. Patent Publication No. US 2009/0149929 entitled “MONITORING THE COOLING OF SUBCUTANEOUS LIPID-RICH CELLS, SUCH AS THE COOLING OF ADIPOSE TISSUE”;

U.S. Provisional Patent Application Ser. No. 60/941,567 entitled “METHODS, APPARATUSES AND SYSTEMS FOR COOLING THE SKIN AND SUBCUTANEOUS TISSUE”;

U.S. Patent Publication No. US 2010/0081971 entitled “TREATMENT PLANNING SYSTEMS AND METHODS FOR BODY CONTOURING APPLICATIONS”;

U.S. patent application Ser. No. 12/275,002 entitled “APPARATUS WITH HYDROPHILIC RESERVOIRS FOR COOLING SUBCUTANEOUS LIPID-RICH CELLS”;

U.S. patent application Ser. No. 12/275,014 entitled “APPARATUS WITH HYDROPHOBIC FILTERS FOR REMOVING HEAT FROM SUBCUTANEOUS LIPID-RICH CELLS”;

U.S. Patent Publication No. US 2010/0152824 entitled “SYSTEMS AND METHODS WITH INTERRUPT/RESUME CAPABILITIES FOR COOLING SUBCUTANEOUS LIPID-RICH CELLS”;

U.S. Patent Publication No. US 2008/0077202 entitled “TISSUE TREATMENT METHODS”; and

U.S. Patent Publication No. US 2010/0280582 entitled “DEVICE, SYSTEM AND METHOD FOR REMOVING HEAT FROM SUBCUTANEOUS LIPID-RICH CELLS.”

### TECHNICAL FIELD

The present application relates generally to treatment devices, systems, and methods for removing heat from subcutaneous lipid-rich tissue. In particular, several embodiments are directed to cryoprotectant compositions or compounds for use with treatment devices to effect heat removal or extraction from subcutaneous lipid-rich tissue.

### BACKGROUND

Excess body fat, or adipose tissue, may be present in various locations of the body, including, for example, the thigh, buttocks, abdomen, knees, back, face, arms, chin, and other areas. Moreover, excess adipose tissue is thought to magnify the unattractive appearance of cellulite, which forms when subcutaneous fat protrudes into the dermis and creates dimples where the skin is attached to underlying structural fibrous strands. Cellulite and excessive amounts of adipose tissue are often considered to be unappealing. Moreover, significant health risks may be associated with higher amounts of excess body fat.

A variety of methods have been used to treat individuals having excess body fat. In many instances, non-invasive removal of excess subcutaneous adipose tissue can eliminate unnecessary recovery time and discomfort associated with invasive procedures such as liposuction. Conventional non-invasive treatments for removing excess body fat typically include topical agents, weight-loss drugs, regular exercise, dieting or a combination of these treatments. One drawback of these treatments is that they may not be effective or even possible under certain circumstances. For example, when a person is physically injured or ill, regular exercise may not be an option. Similarly, weight-loss drugs or topical agents are not an option when they cause an allergic or negative reaction. Furthermore, fat loss in selective areas of a person's body often cannot be achieved using general or systemic weight-loss methods.

Other methods designed to reduce subcutaneous adipose tissue include laser-assisted liposuction and mesotherapy. Newer non-invasive methods include applying radiant energy to subcutaneous lipid-rich cells via, e.g., radio frequency and/or light energy, such as described in U.S. Patent Publication No. US 2006/0036300 and U.S. Pat. No. 5,143,063, or via, e.g., high intensity focused ultrasound (HIFU) radiation such as described in U.S. Pat. Nos. 7,258,674 and 7,347,855. In contrast, methods and devices for non-invasively reducing subcutaneous adipose tissue by cooling are disclosed in U.S. Pat. No. 7,367,341 entitled “METHODS AND DEVICES FOR SELECTIVE DISRUPTION OF FATTY TISSUE BY CONTROLLED COOLING” to Anderson et al. and U.S. Patent Publication No. US 2005/0251120 entitled “METHODS AND DEVICES FOR DETECTION AND CONTROL OF SELECTIVE DISRUPTION OF FATTY TISSUE BY CONTROLLED COOLING” to Anderson et al., the entire disclosures of which are incorporated herein by reference.

### BRIEF DESCRIPTION OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn are not intended to convey any information regarding the actual shape of the particular elements, and have been solely selected for ease of recognition in the drawings.

FIG. 1 is a partially schematic, isometric view of a treatment system for non-invasively removing heat from subcutaneous lipid-rich target areas of a patient in accordance with an embodiment of the disclosure.

FIG. 2 is a schematic cross-sectional view of the treatment device of FIG. 1.

### DETAILED DESCRIPTION

### A. Overview

The present disclosure describes compositions or compounds for use with treatment devices to effect heat removal or extraction from subcutaneous lipid-rich tissue. One embodiment of a composition for use with a system for cooling subcutaneous lipid-rich tissue, for example, comprises a cryoprotectant agent configured to be applied to an interface between a treatment device and skin of a subject. The cryoprotectant agent is configured to be in contact with at least one of the skin of the subject at a target region and a surface of the treatment device. The composition can also include an authentication agent, a lubricating agent, and a thixotropic agent present in, dispersed in, or uniformly dispersed in the cryoprotectant agent. The authentication agent is detectable by at least one of an X-ray fluorescence process, atomic spectrometry techniques (e.g., atomic absorption spectrometry, inductively coupled plasma atomic emission spectrometry), gas chromatography, gas chromatography-mass spectrometry, infrared (IR) spectrometry, and an opto-chemical process. The lubricating agent is configured to increase lubriciousness of the cryoprotectant agent and thereby reduce friction at the interface between the treatment device and the skin of the subject. The thixotropic agent is present in the cryoprotectant agent in an amount sufficient to impart a desired thixotropic property to the cryoprotectant agent.

Another embodiment of the disclosure is directed to a non-freezing cryoprotectant gel for use with a system for cooling subcutaneous lipid-rich cells of a mammal, such as a person. The gel can include at least two of the following additives: (a) a gel authentication additive present in the gel to authenticate an origin of the gel, the gel authentication additive being detectable using at least one of an x-ray fluorescence process, atomic spectrometry, gas chromatography, gas chromatography-mass spectrometry, IR spectrometry, and an opto-chemical process, (b) an additive present in the gel to increase the lubriciousness of the gel, and (c) a pseudoplastic additive in an amount effective to render the gel substantially dimensionally stable unless agitated.

Still another embodiment of the disclosure is directed to a cryoprotectant for use with a system for removing heat from subcutaneous lipid-rich cells of a mammal to protect biological tissues of the mammal from freezing damage during treatment. The cryoprotectant can include, for example, a temperature depressant to provide a freezing point of the cryoprotectant of from about −40° C. to about 0° C., and a thickening agent to provide a viscosity of the cryoprotectant in the range of about 1 cP to about 10,000 cP. The cryoprotectant can also include a pH buffer to maintain a pH in the cryoprotectant in the range of about 3.5 to about 11.5, a humectant, and a surfactant. The cryoprotectant can further include an anti-counterfeiting additive detectable by at least one of an X-ray fluorescence process, atomic spectrometry, gas chromatography, gas chromatography-mass spectrometry, IR spectrometry, and an opto-chemical process. The cryoprotectant also includes a soluble, lubricity additive to provide a desired lubriciousness to the cryoprotectant, and a thixotropic or pseudoplastic additive.

Although the following description provides many specific details of the following examples in a manner sufficient to enable a person skilled in the relevant art to practice, make, and use them, several of the details and advantages described below may not be necessary to practice certain examples and methods of the disclosure. Additionally, the disclosure may include other examples and methods that are within the scope of the claims but are not described in detail.

Reference throughout this specification to “one example,” “an example,” “one embodiment” or “an embodiment” means that a particular feature, structure, or characteristic described in connection with the example is included in at least one example of the present disclosure. Thus, the occurrences of the phrases “in one example,” “in an example,” “one embodiment” or “an embodiment” in various places throughout this specification are not necessarily all referring to the same example. Furthermore, the particular features, structures, routines, steps or characteristics may be combined in any suitable manner in one or more examples of the disclosure. The headings provided herein are for convenience only and are not intended to limit or interpret the scope or meaning of the claimed disclosure.

### A. Suitable Treatment System

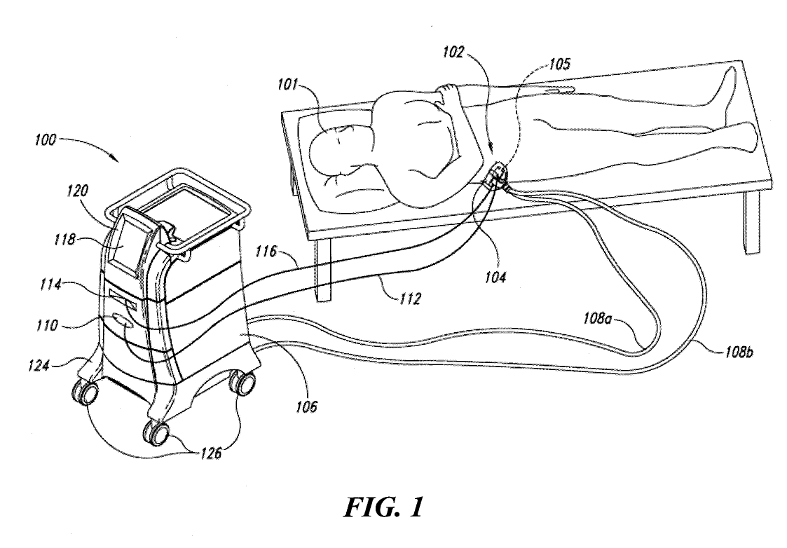

FIG. 1 and the following discussion provide a brief, general description of a suitable treatment system 100 in which aspects of the present technology can be implemented. Those skilled in the relevant art will appreciate that the present technology can be practiced with other systems and treatment protocols, including invasive, minimally invasive, other non-invasive cosmetic or medical treatment systems, and/or combinations of one or more of the above for treating a patient or subject 101. In general, the term “treatment system”, as used generally herein, refers to any of the above system categories of cosmetic or medical treatments as well as any treatment regimes or medical device usage.

The treatment system 100 is suitable for cooling the subcutaneous adipose tissue of the patient or subject 101 in a manner that reduces the volume of the adipose tissue. “Subcutaneous tissue” can include tissue lying beneath the dermis and includes subcutaneous fat or adipose tissue that may be composed primarily of lipid-rich cells, or adipocytes. When cooling subcutaneous tissues to a temperature lower than 37° C., subcutaneous lipid-rich cells can be affected selectively. In general, the epidermis and dermis of the patient 101 have lower amounts of unsaturated fatty acids compared to the underlying lipid-rich cells forming the subcutaneous tissue. Because non-lipid-rich cells usually can withstand colder temperatures better than lipid-rich cells, the subcutaneous lipid-rich cells can be affected selectively without affecting the non-lipid-rich cells in the dermis, epidermis and other surrounding tissue. In some embodiments, the treatment system 100 can apply cooling temperatures to the skin of the patient 101 in a range of about −20° C. to about 20° C. In other embodiments, the cooling temperatures can be from about −20° C. to about 10° C., approximately 0° C. to approximately 20° C., about −15° C. to about 5° C, approximately −5° C. to approximately 15° C., or about −10° C. to about 0° C.

Without being bound by theory, the selective effect of cooling on lipid-rich cells is believed to result in, for example, membrane disruption, cell shrinkage, disabling, destroying, removing, killing, or other methods of lipid-rich cell alteration. Such alteration is believed to stem from one or more mechanisms acting alone or in combination. It is thought that such mechanism(s) trigger an apoptotic cascade, which is believed to be the dominant form of lipid-rich cell death by non-invasive cooling.

Apoptosis, also referred to as “programmed cell death,” is a genetically-induced death mechanism by which cells self-destruct without incurring damage to surrounding tissues. An ordered series of biochemical events induce cells to morphologically change. These changes include cellular blebbing, loss of cell membrane asymmetry and attachment, cell shrinkage, chromatin condensation and chromosomal DNA fragmentation. Injury via an external stimulus, such as cold exposure, is one mechanism that can induce cellular apoptosis in cells. Nagle, W. A., Soloff, B. L., Moss, A. J. Jr., Henle, K. J. “Cultured Chinese Hamster Cells Undergo Apoptosis After Exposure to Cold but Nonfreezing Temperatures” Cryobiology 27, 439-451 (1990).

One aspect of apoptosis, in contrast to cellular necrosis (a traumatic form of cell death causing local inflammation), is that apoptotic cells express and display phagocytic markers on the surface of the cell membrane, thus marking the cells for phagocytosis by, for example, macrophages. As a result, phagocytes can engulf and remove the dying cells (e.g., the lipid-rich cells) without eliciting an immune response. Temperatures that elicit these apoptotic events in lipid-rich cells may contribute to long-lasting and/or permanent reduction and reshaping of subcutaneous adipose tissue.

One mechanism of apoptotic lipid-rich cell death by cooling is believed to involve localized crystallization of lipids within the adipocytes at temperatures that do not induce crystallization in non-lipid-rich cells. The crystallized lipids selectively may injure these cells, inducing apoptosis (and may also induce necrotic death if the crystallized lipids damage or rupture the bi-lipid membrane of the adipocyte). Another mechanism of injury involves the lipid phase transition of those lipids within the cell's bi-lipid membrane, which results in membrane disruption, thereby inducing apoptosis. This mechanism is well-documented for many cell types and may be active when adipocytes, or lipid-rich cells, are cooled. Mazur, P., “Cryobiology: the Freezing of Biological Systems” Science, 68: 939-949 (1970); Quinn, P. J., “A Lipid Phase Separation Model of Low Temperature Damage to Biological Membranes” Cryobiology, 22: 128-147 (1985); Rubinsky, B., “Principles of Low Temperature Preservation” Heart Failure Reviews, 8, 277-284 (2003). Other yet-to-be understood apoptotic mechanisms may exist, based on the relative sensitivity of lipid-rich cells to cooling compared to non-lipid rich cells.

In addition to the apoptotic mechanisms involved in lipid-rich cell death, local cold exposure also is believed to induce lipolysis (i.e., fat metabolism) of lipid-rich cells and has been shown to enhance existing lipolysis which serves to further increase the reduction in subcutaneous lipid-rich cells. Vallerand, A. L., Zamecnik. J., Jones, P. J. H., Jacobs, I. “Cold Stress Increases Lipolysis, FFA Ra and TG/FFA Cycling in Humans” Aviation, Space and Environmental Medicine 70, 42-50 (1999).

In various embodiments, the treatment system 100 includes a controller, a computing device, a data acquisition device, a chiller, and one or more treatment devices. These components can be implemented in various embodiments to apply selected treatment profiles to the patient 101 (e.g., a human or animal) for reducing adipose tissue.

FIG. 1 is a partially schematic, isometric view illustrating one example of a treatment system 100 for non-invasively removing heat from subcutaneous lipid-rich target areas of the patient 101, such as an abdominal area 102 or another suitable area. The system 100 may include a treatment device 104 that engages the target area of the patient 101 and a treatment unit 106 that operate together to cool or otherwise remove heat from the subcutaneous lipid-rich cells of the patient 101. The treatment device 104 can be part of an application system, and the treatment device 104 can have various, configurations, shapes and sizes suitable for different body parts such that heat can be removed from any subcutaneous lipid-rich target area of the patient 101. For example, the treatment device 104 may be designed to treat target areas of the patient's body, such as chin, cheeks, arms, pectoral areas, thighs, calves, buttocks, back, abdomen, “love handles,” and so forth. The treatment device 104 can have a cooling unit 105 that cools a selected area of the patient 101. The system 100 can also include a disposable protective device and a cryoprotectant for cooling the lipid-rich adipose tissue.

As explained in more detail below, a cryoprotectant composition or compound having a freezing point in the range of about −40° C. to about 0° C. is applied to an interface between the treatment device 104 and the skin of the patient or subject 101. As used herein, “cryoprotectant,” “cryoprotectant agent,” and “composition” mean compounds that assist in preventing freezing of non lipid-rich tissue (e.g., dermal tissue) compared to an absence of the compound. The cryoprotectant allows, for example, the treatment device 104 to be pre-cooled prior to being applied to the patient 101 for more efficient treatment. Further, the cryoprotectant can also enable the treatment device 104 to be maintained at a desired temperature while preventing ice from forming on a surface of the treatment device 104, and thus reduces the delay in reapplying the treatment device 104 to the subject. Yet another advantage is that the cryoprotectant may prevent the treatment device 104 from freezing to the skin of the patient or subject 101. Additionally, the cryoprotectant may protect biological tissues of a subject, such as a mammal, from freezing damage (e.g., damage due to ice formation). If the cryoprotectant is hygroscopic, it can adsorb moisture from the atmosphere and/or from the surface of the skin, which might otherwise form ice. The cryoprotectant may also include one or more additives present in the compound and configured to provide selected properties to the compound. Further details regarding cryoprotectants suitable for use with the treatment system 100 are described in greater detail below in Section B.

In the embodiment illustrated in FIG. 1, the treatment device 104 may provide mechanical energy to create a vibratory, massage, and/or pulsatile effect in addition to cooling subcutaneous adipose tissue. Several examples of such devices are described in U.S. Pat. No. 7,367,341 and commonly assigned U.S. Patent Publication No. US 2008/0287839. The treatment device 104, for example, may include one or more actuators that generate a transitory force which is transmitted to the subject. Suitable actuators include motors with eccentric weights, hydraulic motors, electric motors, pneumatic motors, solenoids, other mechanical motors, piezoelectric shakers, and other devices that provide vibratory energy to the treatment site. A single treatment device 104 may have a plurality of different types of actuators in any desired combination. For example, the treatment device 104 may have an eccentric weight actuator (not shown) and a pneumatic motor (not shown) such that different effects may be provided with the same treatment device 104. This arrangement is expected to provide a number of options for differential treatments of lipid rich cells within a single target area or among multiple target areas of patient 101.

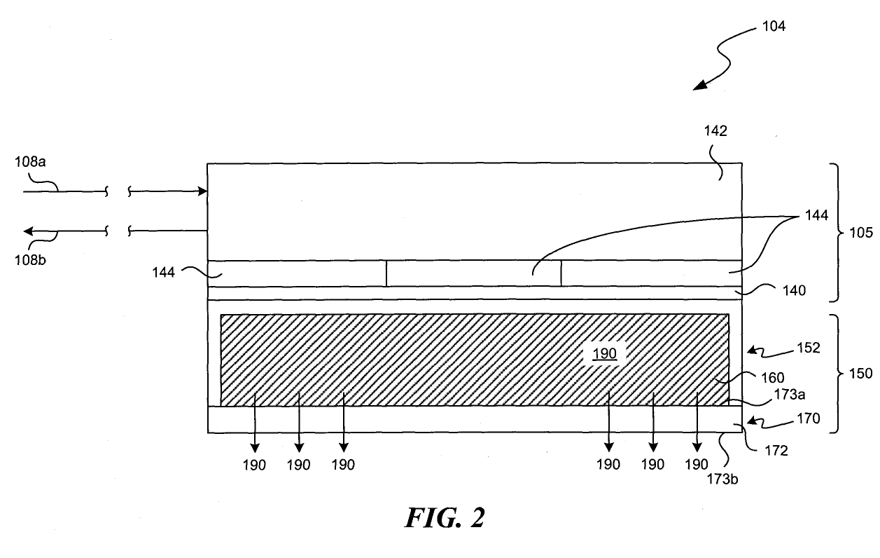

FIG. 2 is an enlarged schematic cross-sectional view of the treatment device 104 of FIG. 1. For purposes of illustration, a number of components of the treatment device 104 are not shown or described. The treatment device 104 includes (a) an interface assembly 150 configured to contact the target area, and (b) the cooling unit 105. In this embodiment, the cooling unit 105 is a component of a cooling system integrated with the treatment device 104. The cooling unit 105 can include a plate 140 having a high thermal conductivity, a coolant chamber 142, and one or more Peltier-type thermoelectric elements 144, such as a plurality of individually controlled thermal segments that create a custom spatial cooling profile and/or a time-varying cooling profile. Each custom treatment profile can include one or more segments, and each segment can include a specified duration, a target temperature, and control parameters for features such as vibration, massage, vacuum, and other treatment modes. Cooling devices having multiple individually controlled heat exchanging units are described, e.g., in commonly assigned U.S. Patent Publication No. US 2008/0077211.

A coolant can circulate through the coolant chamber 142 via supply and return 108a and 108b, respectively, and the thermoelectric elements 144 can selectively heat and/or cool relative to the temperature of the coolant in the coolant chamber 142 to control the temperature over relatively large areas of the cooling plate 140. Other embodiments of the cooling unit 105 do not include the thermoelectric elements 144 such that the coolant chamber 142 extends to the plate 140. In either case, the cooling unit 105 provides a heat sink that cools the interface assembly 120. In still other embodiments, the cooling unit 105 may have a different arrangement and/or different features.

The interface assembly 150 of the treatment device 104 further controls the heat flux through a plurality of smaller zones and delivers a cryoprotectant to the target area. In the illustrated embodiment, the interface assembly 150 includes a cavity 152, a cryoprotectant container 160 that contains a cryoprotectant 190, and an interface element 170 through which the cryoprotectant 190 can flow. The reservoir 160 is configured to provide a continuous or at least an approximately continuous supply of cryoprotectant 190 to the target area during treatment. In other embodiments, the cryoprotectant may be applied directly to an engagement surface of the treatment device 104, the skin of the patient 101, or both, in addition to or in lieu of supplying the cryoprotectant 190 via the container 160.

The interface element 170 can include a contact member 172 having a back side 173a in contact with the cryoprotectant 190 and a front side 173b configured to contact the epidermis of the subject and/or an interface member on the patient's skin. The contact member 172 can be a flexible barrier (e.g., membrane), a mesh, fabric or other suitable material through which the cryoprotectant 190 can flow from the back side 173a to the front side 173b. In other embodiments, the contact member 172 can be a substantially rigid barrier that is thermally conductive and configured to allow the cryoprotectant 190 to pass from the back side 173a to the front side 173b. A rigid contact member, for example, can be a plate with holes or a panel made from a porous metal material. In other embodiments, the interface element 170 can have a different arrangement and/or include different features.

Referring to FIGS. 1 and 2 together, the treatment unit 106 may be a refrigeration unit, a cooling tower, a thermoelectric chiller or cooler or any other device or cooling unit capable of removing heat from a coolant in addition to or in lieu of the cooling unit 105 at the treatment device 104. The treatment unit 106 can be operatively coupled to the treatment device 104 by supply and return fluid lines 108a and 108b that circulate chilled fluid (e.g., a coolant) through the treatment device 104. Alternatively, the treatment unit 106 can circulate warm fluid to the treatment device 104 during periods of warming. Examples of the circulating coolant include water, glycol, synthetic heat transfer fluid, oil, a refrigerant, a cryoprotectant, and/or any other suitable heat-conducting fluid. The fluid lines 108a and 108b may be hoses or other conduits constructed from polyethylene, polyvinyl chloride, polyurethane and/or other materials that can accommodate the particular circulating coolant. Furthermore, one skilled in the art will recognize that there are a number of other cooling technologies that could be used such that the cooling units or coolers of the treatment unit 106 or the treatment device 104 need not be limited to those described herein.

Referring to FIG. 1, the treatment system 100 may further include a power supply 110 and a processing unit 114 operatively coupled to the treatment device 104, the cooling unit 105, and/or the treatment unit 106. In one example, the power supply 110 provides a direct current voltage to a thermoelectric element of the cooling unit 105 to adjust the heat flux over a relatively large area. The processing unit 114 may monitor process parameters via sensors (not shown) placed proximate to the treatment device 104 through power line 116 to, among other things, adjust the heat removal rate based on the process parameters. The processing unit 114 may further monitor process parameters to adjust the cooling unit 105 or other components based on the process parameters.

The processing unit 114 may be in direct electrical communication with treatment device 104 through the electrical line 112 as shown in FIG. 1; alternatively, processing unit 114 may be connected to treatment device via a wireless or an optical communication link. For example, the processing unit 114 may be in electrical communication with a control panel of the treatment device 104, the cooling unit 105, and/or an interface assembly. The processing unit 114 may be any processor, programmable logic controller, distributed control system, and so on. Although the power line 116 and the electrical line 112 are shown in FIG. 1 without any support structure, these lines and other lines including, but not limited to the fluid lines 108a and 108b, may be bundled into or otherwise accompanied by a conduit or the like to protect the lines, enhance user safety and ergonomic comfort, inhibit unwanted motion that could adversely impact the heat transfer rate, provide electrical and thermal insulation, and provide an aesthetic appearance to the treatment system 100. Examples of such a conduit include a flexible polymeric fabric, a composite sheath, an adjustable arm, etc. Such a conduit (not shown) may be designed (via adjustable joints, etc.) to “set” the conduit in place for the treatment of the patient 101.

The treatment system 100 can also include an input device 118 and an output device 120 operatively coupled to the processing unit 114. The input device 118 may be a keyboard (shown schematically in FIG. 1), a mouse, a touch screen, a push button, a switch, a potentiometer, any combination thereof and any other device or devices suitable for accepting user input. The output device 120 may include a display or touch screen, a printer, a medium reader, an audio device, a visual device, any combination thereof and any other device or devices suitable for providing user feedback. In the embodiment of FIG. 1, the input device 118 and the output device 120 may be combined in a single unit, such as a touch screen.

The control panel of the treatment device 104 may include visual indicator devices or controls (lights, numerical displays, etc.) and/or audio indicator devices or controls. The control panel may be a separate component from the input device and/or output device as shown in FIG. 1, or the control panel may be (a) integrated with one or more of the input and output devices 118 and 120, (b) partially integrated with one or more of the input and output devices 118 and 120, (c) at another location, and so on. In this example, the processing unit 114, the power supply 110, the treatment unit 106, the input device 118, and the output device 120 are carried by a rack or cart 124 with wheels 126 for portability. In alternative examples, the processing unit 114 may be contained in, attached to, or integrated with the treatment device 104, the cooling unit 105, and/or an interface assembly. In yet another example, the various components may be fixedly installed at a treatment site. Further details with respect to selected versions of the components and/or operation of the treatment device 104, cooling unit 105, and other components may be found in commonly-assigned U.S. Patent Publication No. US 2008/0287839.

Without being bound by theory, it is believed that effective conductive cooling from the treatment device 104 depends on a number of factors. Examples of factors that impact heat removal or extraction from the skin and related tissue include, for example, the surface area of the treatment unit, the temperature of the interface member, the mechanical energy delivered to the tissue, the distribution of cryoprotectant, and the extent of non-uniformities in the contact between the interface member and the skin. More specifically, upon receiving input to start a treatment protocol, the processing unit 114 can cause the treatment device 104 to cycle through each segment of a prescribed treatment plan. In so doing, the treatment device 104 applies power to one or more cooling segments, such as thermoelectric coolers (e.g., TEC “zones”), to begin a cooling cycle and, for example, activate features or modes such as vibration, massage, vacuum, etc. Using temperature or heat flux sensors, the processing unit 114 determines whether the temperature and/or heat flux at one or more areas of the actuator are sufficiently close to a target temperature or target heat flux. It will be appreciated that while a region of the body (e.g., adipose tissue) has been cooled or heated to the target temperature or by a target heat flux, in actuality that region of the body may be close but not equal to the target temperature, e.g., because of the body's natural heating and cooling variations. Thus, although the treatment system 100 may attempt to heat or cool to the target temperature or by a target heat flux, a sensor may measure a sufficiently close temperature. If the target temperature has not been reached, power can be increased or decreased to change heat flux, as needed, to maintain the target temperature or “set-point.” When the prescribed segment duration expires, the processing unit 114 may apply the temperature and duration indicated in the next treatment profile segment. In some embodiments, temperature can be controlled using a variable other than, or in addition to, power.

The treatment device 104, the cryoprotectant, and/or other components of the treatment system 100 can be included in a kit (not shown) for removing heat from subcutaneous lipid rich cells of the patient 101. The kit can also include instruction documentation containing information regarding how to (a) apply the composition to a target region and/or a heat exchanging surface of the treatment device 104 and (b) reduce a temperature of the target region such that lipid rich cells in the region are affected while preserving non-lipid rich cells proximate to the heat exchanging surface.

### B. Suitable Cryoprotectants

A cryoprotectant suitable to be used in the treatment system 100 of FIG. 1 is a substance that may protect biological tissues of a subject from freezing damage (e.g., damage due to ice formation). The cryoprotectant may contain a temperature depressant along with one or more other components, e.g., a thickening agent, a pH buffer, a humectant, a surfactant, and/or other additives configured to provide selected properties to the compound. The cryoprotectant may be formulated as a non-freezing liquid (e.g., an aqueous solution or a non-aqueous solution), a non-freezing gel, a non-freezing hydrogel, or a non-freezing paste. The cryoprotectant may be hygroscopic, thermally conductive, and is ideally biocompatible. In certain embodiments, the cryoprotectant may be formulated to be ultrasonically acoustic to allow ultrasound to pass through the cryoprotectant, such as a water-based gel described in U.S. Pat. No. 4,002,221 issued to Buchalter and U.S. Pat. No. 4,459,854 issued to Richardson et al., the entire disclosures of which are incorporated herein by reference.

The temperature depressant can include polypropylene glycol (PPG), polyethylene glycol (PEG), propylene glycol, ethylene glycol, dimethyl sulfoxide (DMSO), or other glycols. The temperature depressant may also include ethanol, propanol, iso-propanol, butanol, and/or other suitable alcohol compounds. The temperature depressant may lower the freezing point of a solution (e.g., body fluid) to about 0° C. to −40° C., and more preferably to about −10° C. to −16° C. Certain temperature depressants (e.g., PPG, PEG, etc.) may also be used to improve smoothness of the cryoprotectant and to provide lubrication.

The thickening agent can include carboxyl polyethylene polymer, hydroxyethyl xylose polymer, carboxyl methylcellulose, hydroxyethyl cellulose (HEC), and/or other viscosity modifiers to provide a viscosity in the range of about 1 cP to about 10,000 cP, more preferably in the range of about 4,000 cP to about 8,000 cP, and most preferably from about 5,000 cP to about 7,000 cP. The cryoprotectant with a viscosity in this range may readily adhere to the treatment device, the skin of the subject, and/or the interface between the treatment device and the skin of the subject during treatment. As discussed in greater detail below, the cryoprotectant may also include a thixotropic additive in an amount effective to render the cryoprotectant dimensionally stable unless agitated.

The pH buffer may include cholamine chloride, cetamidoglycine, tricine, glycinamide, bicine, and/or other suitable pH buffers. The pH buffer may help the cryoprotectant to have a consistent pH of about 3.5 to about 11.5, more preferably about 5 to about 9.5, and most preferably about 6 to about 7.5. In certain embodiments, the pH of the cryoprotectant may be close to the pH of the skin of the subject.

The humectant may include glycerin, alkylene glycol, polyalkylene glycol, propylene glycol, glyceryl triacetate, polyols (e.g., sorbitol and/or maltitol), polymeric polyols (e.g., polydextrose), quillaia, lactic acid, and/or urea. The humectant may promote the retention of water to prevent the cryoprotectant from drying out.

The surfactant may include sodium dodecyl sulfate, ammonium lauryl sulfate, sodium lauryl sulfate, alkyl benzene sulfonate, sodium lauryl ether sulfate, and other suitable surfactants. The surfactant may promote easy spreading of the cryoprotectant when an operator applies the cryoprotectant to the treatment device, the skin of the subject, and/or the interface between the treatment device and the skin of the subject during treatment. As described in greater detail below, the cryoprotectant may also include a thixotropic or pseudoplastic additive to further enhance application of the cryoprotectant.

In a particular embodiment, the cryoprotectant may include about 30% polypropylene glycol, about 30% glycerin, and about 40% ethanol by weight. In another embodiment, the cryoprotectant may include about 40% propylene glycol, about 0.8% HEC, and about 59.2% water by weight. In a further embodiment, the cryoprotectant may include about 50% polypropylene glycol, about 40% glycerin, and about 10% ethanol by weight. In yet another embodiment, the cryoprotectant may include about 59.5% water, about 40% propylene, and about 0.5% HEC by weight.

In several embodiments, the cryoprotectant may also include a lubricating additive or agent present in the cryoprotectant and configured to increase lubriciousness of the cryoprotectant and thereby reduce friction at the interface between the treatment device and the skin of the subject. The lubricating agent is soluble in the cryoprotectant and does not change the overall effectiveness or bulk properties of the cryoprotectant. The lubricating agent, for example, can include polyethylene oxide (PEO), polyethylene glycol (PEG), polyacrylamide, or another suitable material. In one specific embodiment, for example, the lubricating agent comprises polyethylene oxide in an amount of from about 0.1% to 1% by weight. In another embodiment, the lubricating agent comprises polyethylene oxide in an amount of about 0.5% by weight. The polyethylene oxide can have a molecular weight in a range of from about 20K to about 5000K. For example, the polyethylene oxide can have a molecular weight of about 20K, 50K, 100K, 400K, 2000K, or 5000K. In other embodiments, however, the lubricating agent can be present in the cryoprotectant in a different amount and/or the lubricating agent can comprise a different material.

Without being bound by theory, the present inventors have discovered that the lubricating additive or agent in the cryoprotectant provides a means of reducing friction at the interface between the patient's skin and the treatment device. This is expected to improve the draw of tissue against the treatment device, thereby providing a more complete and effective treatment. By way of example, in one specific treatment process, an interface member is placed directly over the target area of the patient, and the treatment device 104 with a disposable sleeve or liner is placed in contact with the interface member for treatment. The interface member is a reservoir containing a desired volume of cryoprotectant. The interface member can include, for example, a non-woven cotton fabric pad saturated with the cryoprotectant. Suitable pads include Webril™ pads manufactured by Covidien of Mansfield, Mass. Further details regarding the interface member and associated systems and methods are described in commonly-assigned U.S. Provisional Patent Application No. 61/174,487. In other embodiments, however, the interface member can include other suitable pads or devices.

The efficiency of cooling the tissue can be enhanced by temporarily reducing or eliminating blood flow-through the treatment region using a vacuum or other technique. Applying a vacuum may also pull skin and underlying adipose tissue away from the body that can assist in cooling underlying tissue by increasing the distance between the subcutaneous fat and the relatively well-perfused muscle tissue and by allowing the underlying adipose tissue simultaneously to be cooled from two sides. After applying a vacuum, the treatment device 104 is drawn against the skin of the patient, and the interface member is between the treatment device 104 and the skin of the patient. In some instances, however, the draw of the patient's tissue against the treatment device 104 may be inhibited by the friction between (a) the skin and the interface member, and/or (b) the interface member and the treatment device 104. In either case, inhibiting this draw may negatively affect the overall effectiveness of the treatment. As explained above, however, the lubricating additive or agent dispersed in the cryoprotectant is expected to increase lubriciousness of the cryoprotectant and thereby decrease the friction at the two interfaces. The reduced friction is expected to result in greater efficacy and more complete treatment.

The cryoprotectant may also include an authentication or anti-counterfeiting additive present in the cryoprotectant and used to authenticate an origin of the cryoprotectant. As provided above, the chemistry of the cryoprotectant is important to provide effective and safe treatment. Counterfeit or knock-off compounds produced by unauthorized third parties may not have the appropriate chemistry, and use of such compounds may have significant negative effects on the treatment process. The present inventors have discovered that the incorporation of an authentication or anti-counterfeiting additive in the cryoprotectant can protect the authenticity of the cryoprotectant and help ensure that only authorized cryoprotectant compositions are used with the treatment system 100.

The authentication agent, for instance, can be present in the cryoprotectant in a desired volume sufficient to be detectable by, for instance, an X-ray fluorescence process, atomic spectrometry techniques (e.g., atomic absorption spectrometry, inductively coupled plasma atomic emission spectrometry), gas chromatography, gas chromatography-mass spectrometry, IR spectrometry, and/or an opto-chemical process, but not affect the appearance, viscosity, and functionality of the cryoprotectant. The authentication additive may comprise ZnO, NaCl, CaCl, or other suitable inorganic metal salts. In other embodiments, the authentication additive may be composed of other suitable materials that are detectable using other suitable techniques. For example, in some embodiments the authentication additive may comprise elemental metals (e.g., aluminum) and/or organic materials detectable by, for instance, gas chromatography, gas chromatography-mass spectrometry, or IR spectrometry. The authentication additive can be added to the cryoprotectant during the initial formation of the cryoprotectant compound. In this way, the identity of the agent and the amount of the agent disposed in the cryoprotectant can be controlled to preserve the security of this information and help prevent the production of unauthorized cryoprotectant compounds.

The cryoprotectant can also include a thixotropic or pseudoplastic additive present in the cryoprotectant in an amount sufficient to impart a desired flow property to the cryoprotectant. As described in greater detail below, these additives in the cryoprotectant can render the cryoprotectant dimensionally stable unless agitated (e.g., during application to the patient).

The cryoprotectant including the thixotropic or pseudoplastic additive is generally considered a non-Newtonian fluid. Without being bound by theory, a non-Newtonian fluid is a fluid whose viscosity is variable based on applied stress. The relationship between shear stress and the strain rate is non-linear and, in some cases, can be time-dependent. For example, thixotropic materials (e.g., solder pastes, certain types of inks, quicksand), exhibit decreasing viscosity over time at a constant shear rate, while pseudoplastic or “shear thinning” materials (e.g., toothpaste, ketchup, paint) exhibit decreasing viscosity with increasing shear rate. For purposes of this disclosure, however, the terms “thixotropic additive” or “pseudoplastic additive” both refer to an additive to the cryoprotectant that dimensionally stabilizes the cryoprotectant such that the cryoprotectant exhibits a stable form at rest, but becomes more fluid when agitated.

The thixotropic or pseudoplastic additive can include fumed silica, silicon dioxide, or another suitable material that provides the desired properties to the cryoprotectant. One suitable commercially-available additive, for example, is Aerosil® 200 manufactured by Evonik Industries of Essen, Germany. A variety of other thixotropic or pseudoplastic additives that impart the desired properties to the cryoprotectant may also be used.

The present inventors have discovered that the thixotropic or pseudoplastic additive in the cryoprotectant provides a means of reducing the amount of cryoprotectant needed for each treatment, thereby reducing overall treatment costs. For example, conventional cryoprotectant compounds may be difficult to apply to only the target area and can drip or run down the patient during application and/or treatment. This can result in the loss of significant amounts of the cryoprotectant material during treatment, as well as requiring time-consuming cleanup for the patient and the practitioner. In contrast with conventional compounds, cryoprotectants including the thixotropic or pseudoplastic additive are dimensionally stable and are relatively easy to apply to only specific target areas on the patient. As discussed above, the cryoprotectant will generally only move or become more fluid when agitated (e.g., during the initial application and during treatment). Accordingly, excess amounts of cryoprotectant are not expected to run or drip onto non-target areas of the patient, and treatments are expected to require less total cryoprotectant material as compared with treatments using conventional materials.

One expected advantage of several of the embodiments described above is that an operator may use lower treatment temperatures for selectively affecting lipid-rich cells of the subject without causing freezing damage to the epidermis and/or dermis of the subject. The applied cryoprotectant may lower the freezing point of the skin of the subject or body fluid in the target region to at least reduce the risk of intracellular and/or extracellular ice formation at such low treatment temperatures.

Another expected advantage is that the epidermis and/or dermis of the patient may be continually protected against freezing damage. It is believed that a topically administered cryoprotectant may protect the treatment region of the skin of the subject. After the cryoprotectant is applied to the skin of the subject, the cryoprotectant is believed to enter the epidermis, the dermis, and eventually the blood stream of the subject. The subject's blood stream then may carry the cryoprotectant away from the treatment region. As a result, the cryoprotectant concentration in the treatment region drops, and the freezing point of the subject's affected body fluid increases to heighten the risk of freezing damage. Accordingly, continually supplying the cryoprotectant to the skin of the subject may at least reduce or even prevent such a risk.

Still another expected advantage of several of the embodiments described above is that the additives to the cryoprotectant can provide a variety of desired additional properties to the cryoprotectant material, with minimal or no effect on the chemistry and rheological properties of the cryoprotectant. Accordingly, the additives will not interfere with the ability of the cryoprotectant to protect a patient's biological tissues from freezing.

Unless the context clearly requires otherwise, throughout the description and the claims, the words “comprise,” “comprising,” and the like are to be construed in an inclusive sense as opposed to an exclusive or exhaustive sense; that is to say, in a sense of “including, but not limited to.” Words using the singular or plural number also include the plural or singular number, respectively. When the claims use the word “or” in reference to a list of two or more items, that word covers all of the following interpretations of the word: any of the items in the list, all of the items in the list, and any combination of the items in the list.

The above detailed descriptions of embodiments of the invention are not intended to be exhaustive or to limit the invention to the precise form disclosed above. While specific embodiments of, and examples for, the invention are described above for illustrative purposes, various equivalent modifications are possible within the scope of the invention, as those skilled in the relevant art may recognize. For example, while steps are presented in a given order, alternative embodiments may perform steps in a different order. The various embodiments described herein may be combined to provide further embodiments.

In general, the terms used in the following claims should not be construed to limit the invention to the specific embodiments disclosed in the specification, unless the above detailed description explicitly defines such terms. While certain aspects of the invention are presented below in certain claim forms, the inventors contemplate the various aspects of the invention in any number of claim forms. Accordingly, the inventors reserve the right to add additional claims after filing the application to pursue such additional claim forms for other aspects of the invention.

I/We claim:

1. A composition for use with a system for cooling subcutaneous lipid-rich tissue of a subject having skin, the composition comprising:
a cryoprotectant agent configured to be applied to an interface between a treatment device and the skin of the subject, wherein the cryoprotectant agent is configured to be in contact with at least one of the skin of the subject at a target region and a surface of the treatment device;
an authentication agent present in the cryoprotectant agent, wherein the authentication agent is detectable by at least one of an X-ray fluorescence process, atomic spectrometry, gas chromatography, gas chromatography-mass spectrometry, infrared (IR) spectrometry, and an opto-chemical process;
a lubricating agent present in the cryoprotectant agent and configured to increase lubriciousness of the cryoprotectant agent and reduce friction at the interface between the treatment device and the skin of the subject; and
a thixotropic agent present in the cryoprotectant agent in an amount sufficient to impart a desired thixotropic property to the cryoprotectant agent.

2. The composition of claim 1 wherein the authentication agent comprises a metal additive disposed in the cryoprotectant agent.

3. The composition of claim 1 wherein the authentication agent comprises an inorganic metal salt.

4. The composition of claim 3 wherein the authentication agent comprises at least one of ZnO, NaCl, and CaCl.

5. The composition of claim 1 wherein the authentication agent comprises an organic material.

6. The composition of claim 1 wherein the authentication agent is disposed in the cryoprotectant agent in a desired volume sufficient to be detectable, but not detrimentally affect the appearance, viscosity, and functionality of the cryoprotectant agent.

7. The composition of claim 6 wherein the authentication agent is detectable in the cryoprotectant agent before, during, and/or after treatment.

8. The composition of claim 1 wherein the lubricating agent is soluble in the cryoprotectant agent.

9. The composition of claim 1 wherein the lubricating agent comprises polyethylene oxide.

10. The composition of claim 1 wherein the lubricating agent comprises polyethylene oxide in an amount of from about 0.1% to about 1% by weight.

11. The composition of claim 1 wherein the lubricating agent comprises polyethylene oxide in an amount of from about 0.4% to about 0.6% by weight.

12. The composition of claim 1 wherein the lubricating agent comprises polyethylene oxide in an amount of about 0.5% by weight.

13. The composition of claim 1 wherein the lubricating agent comprises polyethylene oxide having a molecular weight of about 100K, and wherein the polyethylene oxide is present in an amount of from about 0.1% to about 1% by weight.

14. The composition of claim 1 wherein the lubricating agent comprises polyethylene oxide having a molecular weight of about 400K, and wherein the polyethylene oxide is present in an amount of from about 0.1% to about 1% by weight.

15. The composition of claim 1 wherein the lubricating agent comprises polyethylene oxide having a molecular weight of about 2000K, and wherein the polyethylene oxide is present in an amount of from about 0.1% to about 1% by weight.

16. The composition of claim 1 wherein the lubricating agent comprises polyethylene oxide having a molecular weight of about 5000K, and wherein the polyethylene oxide is present in an amount of from about 0.1% to about 1% by weight.

17. The composition of claim 1 wherein the lubricating agent comprises at least one of polypropylene glycol and polyacrylamide.

18. The composition of claim 1 wherein the thixotropic agent comprises at least one of fumed silica and silicon dioxide.

19. The composition of claim 1 wherein the cryoprotectant agent includes at least one of polypropylene glycol, glycol, polyethylene glycol, ethylene glycol, dimethyl sulfoxide, polyvinyl pyridine, calcium magnesium acetate, sodium acetate, ethanol, propanol, and sodium formate.

20. The composition of claim 1 wherein the cryoprotectant agent has a freezing point in the range of approximately −40° C. to approximately 0° C.

21. The composition of claim 1 wherein the cryoprotectant agent has a freezing point equal to or below approximately −10° C.

22. The composition of claim 1 wherein the composition comprises at least one of a non-freezing liquid, non-freezing gel, or non-freezing paste.

23. A non-freezing cryoprotectant gel for use with a system for cooling subcutaneous lipid-rich cells of a mammal, wherein the gel comprises at least two of the following additives: (a) a gel authentication additive present in the gel to authenticate an origin of the gel, the gel authentication additive being detectable using at least one of an x-ray fluorescence process, atomic spectrometry, gas chromatography, gas chromatography-mass spectrometry, IR spectrometry, and an opto-chemical process, (b) an additive present in the gel to increase the lubriciousness of the gel, and (c) a pseudoplastic additive in an amount effective to render the gel substantially dimensionally stable unless agitated.

24. The cryoprotectant gel of claim 23 wherein the gel is configured substantially to cover an interface between a heat exchanging surface of a treatment device and a target region on the mammal, and wherein the gel is configured to thermally couple the treatment device to skin of the mammal at the target region.

25. The cryoprotectant gel of claim 23 wherein the gel authentication additive substantially does not affect a rheological property of the gel.

26. The cryoprotectant gel of claim 23 wherein the additive to increase the lubriciousness of the gel and the thixotropic additive change at least one rheological property of the gel.

27. A cryoprotectant for use with a system for removing heat from subcutaneous lipid-rich cells of a mammal to protect biological tissues of the mammal from freezing damage during treatment, the cryoprotectant comprising:
a temperature depressant to provide a freezing point of the cryoprotectant of from about −40° C. to about 0° C.;
a thickening agent to provide a viscosity of the cryoprotectant in the range of about 1 cP to about 10,000 cP;
a pH buffer to maintain a pH in the cryoprotectant in the range of about 3.5 to about 11.5;
a humectant;
a surfactant;
an anti-counterfeiting additive detectable by at least one of an X-ray fluorescence process, atomic spectrometry, gas chromatography, gas chromatography-mass spectrometry, IR spectrometry, and an opto-chemical process;
a soluble, lubricity additive to provide a desired lubriciousness to the cryoprotectant; and
a thixotropic or pseudoplastic additive.
